(19) United States
(12) Patent Application Publication
(10) Pub. No.: US 2023/0094514 A1
(43) Pub. Date: Mar. 30, 2023

(54) CONTINUOUS FLOW PROCESS FOR THE PRODUCTION OF ACID CHLORIDES

(71) Applicant: The Johns Hopkins University, Baltimore, MD (US)

(72) Inventors: Shirley M. Klimkiewicz, Gaithersburg, MD (US); Evan P. Lloyd, Clarksville, MD (US); Brenden Herrera, Rockville, MD (US); Luke Rogers, Rockville, MD (US)

(21) Appl. No.: 17/489,851

(22) Filed: Sep. 30, 2021

(51) Int. Cl. C07C 51/60; B01J 19/00

(52) U.S. Cl. CPC C07C 51/60; B01J 19/0066; B01J 2219/00162; B01J 2219/00074

(57) ABSTRACT

A continuous flow process (CFP) for the production of an acid chloride includes the following steps: (i) providing or forming a first reactant comprising a chlorine-donating compound; (ii) providing or forming a second reactant comprising a carboxylic acid; (iii) providing a first continuous flow of the first reactant into a reactor at a first flow rate; (iv) providing a second continuous flow of the second reactant into the reactor at a second flow rate; and (v) mixing the first reactant and the second reactant in a portion of the reactor and reacting the first reactant and the second reactant to provide a reaction product comprising an acid chloride.

### STATEMENT OF GOVERNMENTAL INTEREST

This invention was made with Government support under contract number HR00111620029 awarded by the Defense Advanced Research Projects Agency (DARPA). The Government has certain rights in the invention.

### TECHNICAL FIELD

Embodiments of the presently-disclosed invention relate generally to a continuous flow process (CFP) for the production of an acid chloride, in which the process includes the continuous reaction of a chlorine-donating compound and a carboxylic acid in a reactor. Systems for the continuous flow production of an acid chloride are also provided.

### BACKGROUND

Acid chlorides are a particularly important class of compounds as acid chlorides are often times used as reactants or intermediates for the synthesis of a variety of compounds including industrial chemicals and pharmaceuticals. Acid chlorides, however, are fairly reactive with water, such as moisture in the air, and tend to degrade with time. For instance, acid chlorides tend to undesirably degrade during storage prior to use. Accordingly, an on-demand preparation of acid chlorides may be of particular interest, such as at a site of remote location (e.g., field, aquatic ship, etc.).

### BRIEF SUMMARY

Non-limiting, example embodiments include a continuous flow process (CFP) for the production of an acid chloride is provided, in which the process includes the following steps: (i) providing or forming a first reactant comprising a chlorine-donating compound; (ii) providing or forming a second reactant comprising a carboxylic acid; (iii) providing a first continuous flow of the first reactant into a reactor at a first flow rate; (iv) providing a second continuous flow of the second reactant into the reactor at a second flow rate; and (v) mixing the first reactant and the second reactant in a portion of the reactor and reacting the first reactant and the second reactant to provide a reaction product comprising an acid chloride.

In another example embodiment, a system for a continuous flow production of an acid chloride includes the following: (i) a first reactant housed in a first vessel, in which the first reactant comprises a chlorine-donating compound; (ii) a second reactant housed in a second vessel, in which the second reactant comprises a carboxylic acid; (iii) a reactor, in which the first vessel is operatively connected to the reactor via a first conduit and the second vessel is operatively connected to the reactor via a second conduit; and (iv) a product vessel operatively connected to an outlet of the reactor.

### BRIEF DESCRIPTION OF THE DRAWINGS

Non-limiting, example embodiments will be described more fully hereinafter with reference to the accompanying drawings, in which some, but not all embodiments are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates a schematic for the continuous flow production of an acid chloride utilizing a plug flow reactor in accordance with certain embodiments of the invention;

FIG. 2 illustrates a schematic for the continuous flow production of an acid chloride utilizing a continuous-stirred tank reactor in accordance with certain embodiments of the invention; and

FIG. 3 illustrates a schematic for the continuous flow production of an acid chloride in a plug flow reactor in accordance with certain embodiments of the invention.

### DETAILED DESCRIPTION

Non-limiting, embodiments of the invention now will be described more fully hereinafter with reference to the accompanying drawings, in which some, but not all embodiments of the invention are shown. Indeed, the present invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

Example embodiments of the present invention relate generally to the continuous production of acid chlorides utilizing a continuous flow chemistry, in which a chlorine-donating compound is reacted with a carboxylic acid. In accordance with certain embodiments of the invention, certain processes described and disclosed herein enable on-demand production of a variety of acid chlorides. In this regard, the processes may be performed in remote locations (e.g., land outposts, aquatic ships, aerospace vehicles, etc.) where a particular acid chloride may be desired. For example, continuous production of a particular acid halide may provide on-demand production of the acid halide, which may be used as a reagent and/or intermediate compound in the synthesis of a final compound (e.g., industrial chemical or pharmaceutical compound).

Certain embodiments according to the invention provide a continuous flow process (CFP) for the production of an acid chloride is provided, in which the process includes the following steps: (i) providing or forming a first reactant comprising a chlorine-donating compound; (ii) providing or forming a second reactant comprising a carboxylic acid; (iii) providing a first continuous flow of the first reactant into a reactor at a first flow rate; (iv) providing a second continuous flow of the second reactant into the reactor at a second flow rate; and (v) mixing the first reactant and the second reactant in a portion of the reactor and reacting the first reactant and the second reactant to provide a reaction product comprising an acid chloride.

In accordance with certain embodiments of the invention, the first reactant comprises a chlorine-donating compound that provides one or more chlorine atoms for the replacement of one or more hydroxyl groups of a carboxylic acid upon reaction. Although the particular chlorine-donating compound is not necessarily limited, non-limiting examples of chlorine-donating compounds may include phosphorous(V) chloride (PCl₅), phosphorous(III) chloride (PCl₃), sulfur dichloride oxide (SOCl₂), oxalyl chloride (C₂Cl₂O₂), hydrogen chloride (HCl), or any combination thereof.

In accordance with certain embodiments of the invention, the first reactant comprising the chlorine-donating compound may be provided in liquid form or gaseous form. In certain example embodiments, for example, the first reactant may be provided in a liquid form as a first neat reactant. That is, the first reactant comprising the chlorine-donating compound may be provided in a liquid form/phase, without any solvent. Alternatively, the first reactant comprising the chlorine-donating compound may be provided in liquid form/phase as part of a mixture of the chlorine-donating compound and a solvent. In accordance with certain embodiments of the invention, the chlorine-donating compound may be at least partially miscible in the solvent. In accordance with certain embodiments of the invention, the chlorine-donating compound may be completely miscible in the solvent (i.e., the chlorine-containing compound is completely dissolved in the solvent). The solvent may comprise an aqueous solvent or an organic solvent or mixture of organic solvents. For example, the solvent may comprise dichloromethane, 1,2-dichloroethane, 1,4-dioxane, diethyl ether, ethyl acetate, tetrahydrofuran, toluene, acetonitrile, dimethylformamide, or any combination thereof.

In accordance with certain embodiments of the invention, the carboxylic acid of the second reactant may comprise an alkanoic acid (e.g., containing only carbon, hydrogen and oxygen atoms) or a benzoic acid. For example, the carboxylic acid may comprise an alkanoic acid having from 3 to about 20 carbon atoms, such as at least about any of the following: 3, 4, 5, 6, 7, 8, 9, and 10 carbon atoms, and/or at most about any of the following: 20, 19, 18, 17, 16, 15, 14, 13, 12, 11, and 10 carbon atoms. Additionally or alternatively, the carboxylic acid may comprise a benzoic acid, such as any benzoic acid that is defined by the presence of a carboxylate directly attached to a phenyl ring. By way of example only, the benzoic acid may have from 6 to about 50 carbon atoms, such as at least about any of the following: 6, 8, 10, 12, 15, 18, 20, 22, 24, and 25 carbon atoms, and/or at most about any of the following: 50, 48, 46, 44, 42, 40, 38, 36, 35, 34, 32, 30, 28, 26, 25, 24, 22, and 20 carbon atoms. Additionally or alternatively, the carboxylic acid may comprise from 1 to about 6 carboxyl groups, such as at least about any of the following: 1, 2, and 3 carboxyl groups, and/or at most about any of the following: 6, 5, 4, and 3 carboxyl groups.

In accordance with certain embodiments of the invention, non-limiting examples of suitable carboxylic acids include propionic acid, 2-chloroacetic acid, phenylacetic acid, 4-chlorobenzoic acid, 2-fluorobenzoic acid, acrylic acid, butyric acid, 4-amino benzoic acid, acetyl salicylic acid, ethyl-l-hexanoic acid, formic acid anthralic acid, citric acid, maleic acid, malonic acid, 3-nitrobenzoic acid, oxalic acid, 4-pentenoic acid, tartaric acid, p-toluenesulfonic acid, phenyacetic acid, methanesulfonic acid, terephthalic acid, trimesic acid, and tannic acid.

In accordance with certain embodiments of the invention, mixing the first reactant and the second reactant in a portion of the reactor and reacting the first reactant and the second reactant to provide a reaction product comprising an acid chloride comprises continuously supplying the first reactant and the second reactant at a first molar equivalent ratio between the first reactant and the second reactant from about 0.25:1 to about 4:1, such as at least about any of the following: 0.25:1, 0.33:1, 0.5:1, 0.66:1, 1:1, 1.25:1, 1.5:1, 1.6:1, 1.8:1, and 2:1, and/or at most about any of the following: 4:1, 3.8:1, 3.6:1, 3.4:1, 3.2:1, 3:1, 2.8:1, 2.6:1, 2.4:1, 2.2:1, and 2:1. In accordance with certain embodiments of the invention, mixing the first reactant and the second reactant in a portion of the reactor and reacting the first reactant and the second reactant to provide a reaction product comprising an acid chloride comprises continuously supplying the second reactant and the first reactant at a second molar equivalent ratio between the second reactant and the first reactant from about 0.25:1 to about 4:1, such as at least about any of the following: 0.25:1, 0.33:1, 0.5:1, 0.66:1, 1:1, 1.25:1, 1.5:1, 1.6:1, 1.8:1, and 2:1, and/or at most about any of the following: 4:1, 3.8:1, 3.6:1, 3.4:1, 3.2:1, 3:1, 2.8:1, 2.6:1, 2.4:1, 2.2:1, and 2:1. In this regard, the first reactant and the second reactant may be mixed and/or reacted in a stoichiometric relationship or one of the reactants may be provided in excess (e.g., a greater equivalent compared to the other reactant).

In accordance with certain embodiments of the invention, the first molar equivalent ratio or the second molar equivalent ratio may be achieved by selecting a first flow rate of the first continuous flow of the first reactant and selecting a second flow rate of the second continuous flow of the second reactant based on the a first Molarity of the first reactant and a second Molarity of the second reactant.

In accordance with certain embodiments of the invention, the reactor may comprise a plug flow reactor (PFR), a perfectly mixed flow reactor, a continuously-stirred tank reactor (CSTR), or a tube-in-tube reactor (TTR).

For example, the reactor may comprise a PFR (e.g., a tube, pipe, or conduit) in which the first continuous flow and the second continuous flow enter the PFR at a mixing location that is proximate a first end of the PFR. In accordance with certain embodiments of the invention, the mixing location may comprise a T-shaped junction or a Y-shaped junction that operatively connects the first continuous flow and the second continuous flow to the PFR. For example, the first continuous flow may enter a first port of the junction and the second continuous flow may enter a second port of the junction while a third port of the junction may be connected to the first end of the PFR. In this regard, the first continuous flow and the second continuous flow may be thoroughly mixed within the junction to provide a reactant mixture of the first reactant and the second reactant, which will flow through the PFR and react to form an acid chloride (e.g., a reaction product) that exits the second end of the PFR.

A PFR, sometimes called continuous tubular reactor (CTR) or piston flow reactors, may comprise a reactor system provides a chemical reaction in continuous, flowing systems of often times cylindrical geometry. In this regard, fluid (e.g., the reaction mixture) going through a PFR may be modeled as flowing through the reactor as a series of infinitely thin coherent “plugs”, each with a generally uniform composition, traveling in the axial direction of the reactor, with each plug having a different composition from the ones before and after it. As a plug flows through a PFR, the fluid (e.g., the reaction mixture) should preferably be thoroughly mixed in the radial direction but not as significantly mixed in the axial direction (forwards or backwards). Conceptually, each plug of differential volume may be considered as a separate entity, effectively an infinitesimally small continuous stirred tank reactor, limiting to zero volume. As it flows down the tubular PFR, the residence time of the plug is a function of its position in the reactor.

In accordance with certain embodiments of the invention, the PFR or a conduit connected to the second end of the PFR (e.g., located proximate a second end of the PFR) may comprise a back-pressure valve (e.g., a back-pressure regulator). In this regard, the CFP may comprise maintaining a pre-selected pressure within the PFR via operation of the back-pressure valve. For example, the pre-selected pressure may be determined based on volatility properties (e.g., flash/boiling points) of the first reactant and/or the second reactant at various pressures to eliminate a gaseous escape of either the first reactant or the second reactant from the PFR. In this regard, operation of the back-pressure valve may be adjusted in a manner to prevent the formation and/or escape of one or both reactants as such operational circumstances may prevent the efficient reaction of the reactants and provide a low yield of the desired acid chloride. In accordance with certain embodiments of the invention, the pre-selected pressure may be selected on the flash/boiling points of the reactants at a given operation temperature (e.g., temperature at which the reaction takes place in the reactor) to ensure that neither of the reactants change from a liquid phase to a gas phase. In accordance with certain embodiments of the invention, the preselected pressure may range from about 0 to about 300 psig, such as at least about any of the following: 0, 25, 50, 75, 100, 125, and 150 psig, and/or at most about any of the following: 300, 275, 250, 225, 200, 175, and 150 psig.

The PFR, in accordance with certain embodiments of the invention, may comprise one or more static mixers disposed along at least a portion of a length of the PFR. Static mixers are motionless mixing devices that allow for the inline continuous blending of fluids within a pipeline (e.g., a PFR). With no moving parts, static mixers utilize the energy of the flow stream to generate consistent, cost-effective, and reliable mixing (e.g., in the radial direction). In this regard, the one or more static mixers may facilitate process goals of a continuous reactor system in which the reaction product is homogeneous with regard to degree of reaction achieved, viscosity, and operating temperature. For such operating conditions to be achieved, all the material (e.g., the reactant mixture of the first reactant and the second reactant) within the reactor (e.g., PFR) should ideally be well mixed and have the same residence time with in the reactor (e.g., plug flow). The addition of one or more static mixers inside the PFR (e.g., a tubular reactor) may improve the degree of radial mixing to facilitate achievement of plug flow conditions (e.g., all the material processed through the reactor essentially has the same residence time within the reactor so that the reaction product exiting the reactor has witnessed the same reaction conditions of reactive species. Additionally or alternatively, the inside diameter of the PFR may be selected to generate a larger Reynolds number, such as to achieve a turbulent flow regime.

In accordance with certain embodiments of the invention, the PFR may include a temperature control loop configured to maintain a pre-selected temperature (e.g., operating temperature at which the reaction occurs) along at least a portion of a length of the PFR. Additionally or alternatively, the first reactant and/or the second reactant may enter the PFR at the pre-selected temperature. In this regard, for example, the first reactant and the second reactant may enter the PFR at or near the pre-selected temperature while the temperature control loop may provide heat to the PFR in a manner to maintain the reactant mixture at the pre-selected temperature to facilitate efficient conversion the reactants to the desired acid chloride. For example, the temperature control loop may comprise a heated jacket surrounding at least a portion of the PFR, in which a heating medium may be circulated through the heated jacket on an as-needed basis (e.g., as a function the actual operating temperature in the PFR relative to the pre-selected temperature).

In accordance with certain embodiments of the invention, the pre-selected temperature may comprise from about 20° C. to about 150° C., such as at least about any of the following: 20, 25, 30, 40, 50, 60, 70, and 75° C., and/or at most about any of the following: 150, 140, 130, 120, 110, 100, 90, 80, and 75° C.

In accordance with certain embodiments of the invention, reacting the first reactant and the second reactant (e.g., a reactant mixture that moves through the reactor) is conducted for a given timeframe within the reactor, namely a residence time. For example, the time for a first group of reactant molecules entering the reactor, traveling through the reactor (e.g. unreacted or reacted), and exiting the reactor (e.g., unreacted or reacted), and exiting the reactor (e.g., unreacted or converted to an acid chloride) may be defined as the residence time. In accordance with certain embodiments of the invention, the residence time may comprise from about 1 to about 120 minutes, such as at least about any of the following: 1, 2, 3, 4, 5, 6, 8, 10, 15, 20, 30, 40, 50, and 60 minutes, and/or at most about any of the following: 120, 110, 100, 90, 80, 70, and 60 minutes.

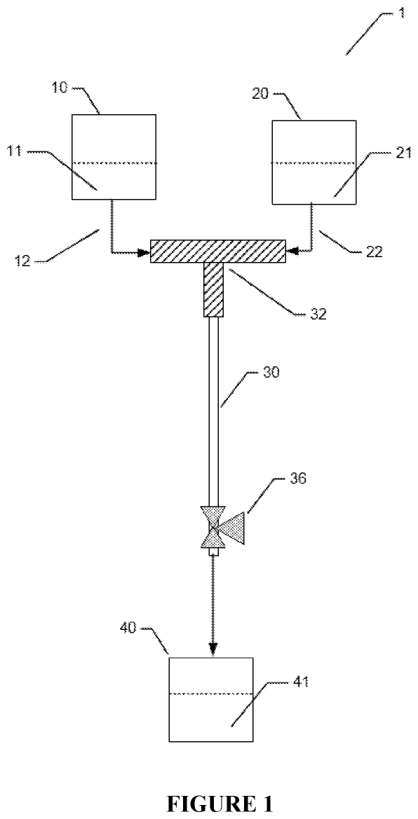

FIG. 1 illustrates a schematic for the continuous flow production (e.g., CFP 1) of an acid chloride utilizing a plug flow reactor 30 in accordance with certain embodiments of the invention. As shown in FIG. 1, the CFP may include a first vessel 10 housing a first reactant 11 comprising a chlorine-donating compound (e.g., contains one or more chlorine atoms that may replace a hydroxyl group of a carboxylic acid) and a second vessel 20 housing a second reactant 21 comprising a carboxylic acid. A first continuous flow 12 of the first reactant 11 and a second continuous flow 22 of the second reactant 21 are formed and conveyed to respective ports of a junction 32 in which the first reactant 11 and the second reactant 21 are continuously mixed to form a reactant mixture and exits the junction via a third port that is operatively connected to a first end of a reactor 30 (e.g., a tube reactor or PFR). As shown in FIG. 1, the reactor 30 (or alternatively a conduit connected to a second end of the reactor) may include a back-pressure valve 36 or pressure regulator configured to be operated in a manner to maintain a pre-selected pressure within the reactor 30. A product vessel 40 may be operatively connected to the second end of the reactor 30 in which the reaction product 41 (e.g., acid chloride) is deposited.

In accordance with certain embodiments of the invention, the reactor may comprise a CSTR including (i) a first inlet operatively connected to the first continuous flow, (ii) a second inlet operatively connected to the second continuous flow, (iii) a product outlet, and (iv) an agitator. A CSTR utilizes a continuous agitated-tank reactor in which the reactants (e.g., the first reactant and the second reactant) are continuously fed to the reactor at a desired rate, while a product stream is continuously pulled or removed from the reactor. Ideally, the reactants (e.g., the first reactant and the second reactant) are quickly (e.g., instantaneously) and uniformly mixed throughout the reactor upon entry. Consequently, the output composition (e.g., product stream including the desired acid chloride) is substantially identical or identical to the composition of the material inside the reactor, which may be a function of residence time and reaction rate. deposited.

In accordance with certain embodiments of the invention, the CSTR may further comprise a gas inlet operatively connected to a gas supply. For example, the gas supply may comprise an inert gas, such as nitrogen gas, which may be added to a head-space of the CSTR in a manner to control the pressure within the CSTR. Accordingly, the gas inlet may further comprise a pressure-control valve or regulator configured to maintain a pre-selected pressure within the CSTR. In accordance with certain embodiments of the invention, the CFP may comprise maintaining a pre-selected pressure within the CSTR via operation of the pressure-control valve that provides more or less gas into the CSTR to increase and/or maintain the pressure within the CSTR. For example, the pre-selected pressure may be determined based on volatility properties (e.g., flash/boiling points) of the first reactant and/or the second reactant at various pressures to eliminate a gaseous escape of either the first reactant or the second reactant from the CSTR. In this regard, operation of the pressure-control valve may be adjusted in a manner to prevent the formation and/or escape of one or both reactants as such operational circumstances may prevent the efficient reaction of the reactants and provide a low yield of the desired acid chloride. In accordance with certain embodiments of the invention, the pre-selected pressure may be selected on the flash/boiling points of the reactants at a given operation temperature (e.g., temperature at which the reaction takes place in the reactor) to ensure that neither of the reactants change from a liquid phase to a gas phase. In accordance with certain embodiments of the invention, the preselected pressure may range from about 0 to about 300 psig, such as at least about any of the following: 0, 25, 50, 75, 100, 125, and 150 psig, and/or at most about any of the following: 300, 275, 250, 225, 200, 175, and 150 psig.

In accordance with certain embodiments of the invention, the CSTR may further comprises a temperature control loop configured to maintain a pre-selected temperature of a reactant mixture within the CSTR (e.g., operating temperature at which the reaction occurs). For example the temperature control loop may comprise a heated jacket surrounding at least a portion of the CSTR and/or a heated coil housed within the CSTR. In this regard, the heated medium may be passed through the heated jacket and/or the heated coil on an as-needed basis (e.g., as a function the actual operating temperature in the CSTR relative to the pre-selected temperature). Additionally or alternatively, the first reactant and/or the second reactant may enter the CSTR at or near the pre-selected temperature. In this regard, for example, the first reactant and the second reactant may enter the CSR at or near the pre-selected temperature while the temperature control loop may provide heat to the CSTR in a manner to maintain the reactant mixture in the CSTR at the pre-selected temperature to facilitate efficient conversion the reactants to the desired acid chloride.

In accordance with certain embodiments of the invention, the pre-selected temperature may comprise from about 20° C. to about 150° C., such as at least about any of the following: 20, 25, 30, 40, 50, 60, 70, and 75° C., and/or at most about any of the following: 150, 140, 130, 120, 110, 100, 90, 80, and 75° C.

In accordance with certain embodiments of the invention, the CFP may further comprise adjusting one or more of the following: (i) a continuous product flow leaving the CSTR via the product outlet; (ii) the first flow rate of the first continuous flow; (iii) the second flow rate of the second continuous flow; (iv) an agitation rate within the CSTR; (v) a pressure within the CSTR; and (vi) a temperature within the CSTR, in response to a concentration of the acid chloride in the continuous product flow. For example, a concentration of the acid chloride in the continuous product flow drops below a pre-defined assay or conversion percentage, one or more the foregoing operational parameters may be adjusted in response to the drop in acid chloride concentration. For instance, the CFP may comprise increasing the pressure and/or reducing the temperature of the reactant mixture within the CSTR to reduce or eliminate off-gassing of one of the reactants in response to the concentration of the acid chloride in the continuous product flow dropping below a pre-defined assay or conversion percentage. Additionally or alternatively, the relative flow rates between the first continuous flow and the second continuous flow may be adjusted to provide an excess of one reactant relative to another.

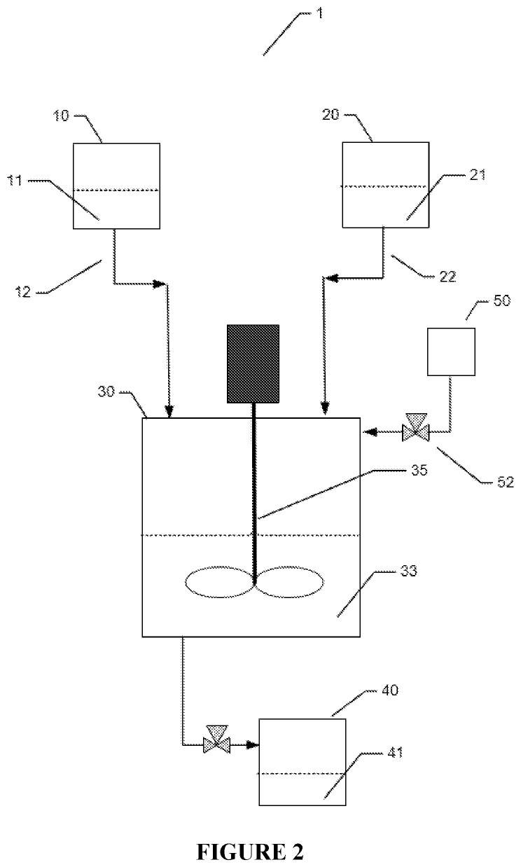

FIG. 2 illustrates a schematic for the continuous flow production (CFP) 1 of an acid chloride utilizing a CSTR in accordance with certain embodiments of the invention. As shown in FIG. 2, the CFP may include a first vessel 10 housing a first reactant 11 comprising a chlorine-donating compound (e.g., contains one or more chlorine atoms that may replace a hydroxyl group of a carboxylic acid) and a second vessel 20 housing a second reactant 21 comprising a carboxylic acid. A first continuous flow 12 of the first reactant 11 and a second continuous flow 22 of the second reactant 21 are formed and conveyed to respective inlets to a CSTR 30 in which the first reactant 11 and the second reactant 21 are continuously mixed via an agitator 35 to form a reactant mixture 33. A continuous product stream comprising a desired acid chloride produced by the reaction product of the first reactant and the second reactant may be deposited in a product vessel 40 that may house the final product 41 comprising the acid chloride of interest. FIG. 2 also shows a gas source 50 configured to supply gas, such as an inert gas, to the CSTR, in which a pressure control valve or regulator 52 may be used to control and/or maintain a desired pressure within the CSTR.

In accordance with certain embodiments of the invention, the reactor may comprise a TTR, in which the first continuous flow passes through an inner tube comprising a semi-permeable membrane that is permeable to gases but impermeable to liquids, and the second continuous flow passes through an outer tube that surrounds the inner tube. For example, the first continuous flow may comprise a gas, such as gaseous HCl that continuously permeates through the semi-permeable membrane and into the second continuous flow comprising the carboxylic acid in initiate reaction for the formation of a desired acid chloride.

In another aspect, the present invention provides a system for a continuous flow production of an acid chloride, in which the system includes the following: (i) a first reactant housed in a first vessel, in which the first reactant comprises a chlorine-donating compound; (ii) a second reactant housed in a second vessel, in which the second reactant comprises a carboxylic acid; (iii) a reactor (e.g., any of the reactor disclosed and described herein), in which the first vessel is operatively connected to the reactor via a first conduit and the second vessel is operatively connected to the reactor via a second conduit; and (iv) a product vessel operatively connected to an outlet of the reactor. As noted previously, systems in accordance with certain embodiments of the invention may also include a pressure-control valve or pressure regulator configured to maintain a pre-selected pressure within the reactor. Additionally or alternatively, the system may comprise one or more of the following: a first heating device configured to supply heat to the first vessel the second vessel or both. In some embodiments, the system may comprise a second heating device configured to supply heat to the second vessel. As noted previously, the system may comprise a third heating device configured to supply heat to at least a portion of the reactor and/or contents within the reactor. In accordance with certain embodiments of the invention, the system may further comprise a first pump configured to transfer the first reactant from the first vessel, through the first conduit, and into the reactor and/or a second pump configured to transfer the second reactant from the second vessel, through the second conduit, and into the reactor.

### EXAMPLES

The present disclosure is further illustrated by the following examples, which in no way should be construed as being limiting. That is, the specific features described in the following examples are merely illustrative and not limiting.

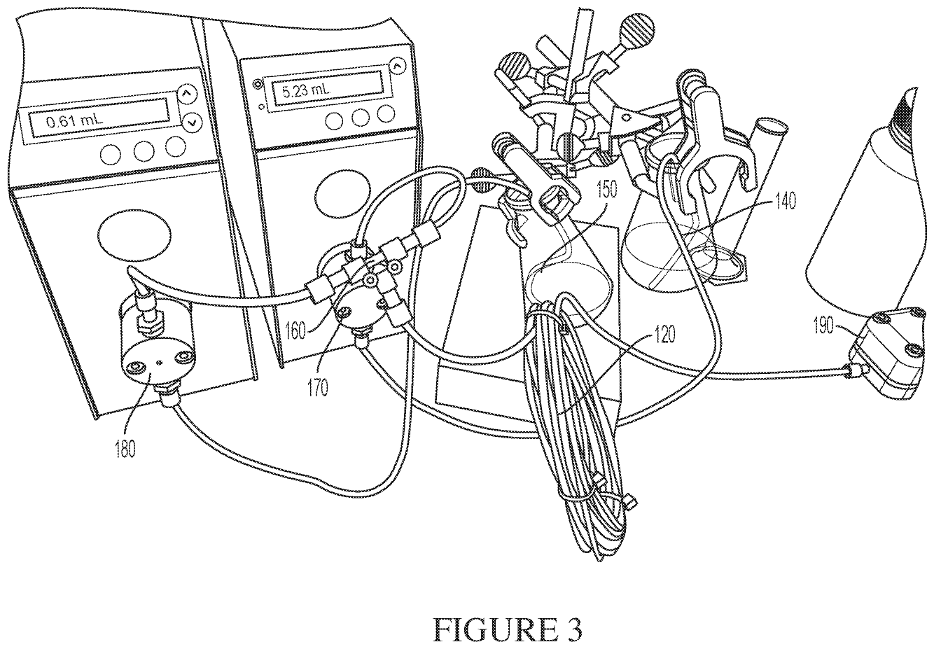

As shown in FIG. 3, a reaction system was assembled that include a hollow-tube reactor (e.g., tube) 120 was operatively connected to a first reactant 140 comprising a chlorine-containing compound and a second reactant 150 comprising a carboxylic acid via a T-shaped junction 160. The first reactant was conveyed into the T-shaped junction via a first pump 170 and the second reactant was conveyed into the T-shaped junction via a second pump 180. A back-pressure regulator 190 was provided at the end of the hollow-tube reactor. The first reactant and the second reactant were continuously reacted in the hollow-tube reactor with a reaction product being collected for analysis of acid chloride production. The hollow-tube reactor had a volume of 15 ml, in which the PFA tube was 25 feet long and had an inside diameter of 1/16″.

### Example 1

Utilizing the reaction system shown in FIG. 3, a continuous flow process for the production of propionyl chloride from the continuous reaction of neat PCl₃ (e.g., the first reactant) with propionic acid (e.g., the second reactant). The neat PCl₃ was pumped at a rate of 0.9 ml/min into the reactor and the propionic acid was pumped at a rate of 2 ml/min into the reactor. A pressure of 50 psig was maintained within the hollow-tube reactor, while the reaction was carried out at a temperature of 75° C. This reaction scheme was based on the following equivalents: 0.4 eq. of PCl₃: 1.0 eq. propionic acid. The residence time for the reaction was 5 minutes. This process provided a 92% conversion.

### Example 2

Utilizing the reaction system shown in FIG. 3, a continuous flow process for the production of 2-fluorobenzoyl chloride from the continuous reaction of neat oxalyl chloride (e.g., the first reactant) with 2-fluorobenzoic acid dissolved in 1,4-dioxane with dimethylformamide (DMF) (e.g., the second reactant). In particular, the second reactant included 50 g of 2-fluorobenzoic acid dissolved in 100 ml of 1,4-dioxane with 2.75 ml of DMF for a total volume of 145 ml. The neat oxalyl chloride was pumped at a rate of 1.23 ml/min into the reactor and the second reactant was pumped at a rate of 6 ml/min into the reactor. A pressure of 250 psig was maintained within the hollow-tube reactor, while the reaction was carried out at a temperature of 30° C. This reaction scheme was based on the following equivalents: 1.0 eq. of oxalyl chloride: 1.0 eq. 2-fluorobenzoic acid: 0.1 eq. DMF. The residence time for the reaction was about 2 minutes. This process provided a conversion percentage above 90%.

### Example Set 3

Utilizing the reaction system shown in FIG. 3, a variety of reaction schemes utilizing different chlorine-donating compounds and different carboxylic acids were investigated for acid chloride production. Table 1 provides a summary of these reactions.

TABLE 1

| Acid | Reaction System | Avg. % Conversion |
|---|---|---|
| propionic acid | neat PCl3 | 79.2 |
| | oxalyl chloride/2M in DCM | 64.8 |
| butyric acid | neat PCl3 | 81.6 |
| | oxalyl chloride/2M in DCM | 80.0 |
| phenylacetic acid | neat PCl3 | 74.9 |
| | oxalyl chloride/2M in DCM | 84.7 |
| p-toluenesulfonic acid | neat PCl3 | — |
| | oxalyl chloride/2M in DCM | — |
| methanesuflonic acid | neat PCl3 | 36.8 |
| | oxalyl chloride/2M in DCM | 55.2 |
| 4-pentenoic acid | neat PCl3 | — |
| | oxalyl chloride/2M in DCM | — |
| acrylic acid | neat PCl3 | — |
| | oxalyl chloride/2M in DCM | — |
| 2-ethylhexanoic acid | neat PCl3 | 75.2 |
| | oxalyl chloride/2M in DCM | — |
| malonic acid | oxalyl chloride/2M in DCM | — |
| 3-nitrobenzoic acid | oxalyl chloride/2M in DCM | — |
| trimesic acid | oxalyl chloride/2M in DCM | — |
| terephtalic acid | oxalyl chloride/2M in DCM | — |

These and other modifications and variations to embodiments of the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

What is claimed is:

1. A continuous flow process (CFP) for production of an acid chloride, the CFP comprising:
(i) providing or forming a first reactant comprising a chlorine-donating compound;
(ii) providing or forming a second reactant comprising a carboxylic acid;
(iii) providing a first continuous flow of the first reactant into a reactor at a first flow rate;
(iv) providing a second continuous flow of the second reactant into the reactor at a second flow rate; and
(v) mixing the first reactant and the second reactant in a portion of the reactor and reacting the first reactant and the second reactant to provide a reaction product comprising an acid chloride.

2. The CFP of claim 1, wherein the first reactant comprises phosphorous(V) chloride (PCl₅), phosphorous(III) chloride (PCl₃), sulfur dichloride oxide (SOCl₂), oxalyl chloride (C₂Cl₂O₂), hydrogen chloride (HCl), or any combination thereof.

3. The CFP of claim 1, wherein the first reactant is provided in liquid form and is provided as a first neat reactant.

4. The CFP of claim 1, wherein the first reactant is provided in liquid form and is provided as a mixture of the chlorine-donating compound and a solvent.

5. The CFP of claim 1, wherein the carboxylic acid of the second reactant comprises a alkanoic acid or a benzoic acid.

6. The CFP of claim 5, wherein the carboxylic acid is an alkanoic acid having from 3 to about 20 carbon atoms.

7. The CFP of claim 5, wherein the carboxylic acid is a benzoic acid having from 6 to about 50 carbon atoms.

8. The CFP of claim 5, wherein the carboxylic acid comprises from 1 to 6 carboxyl groups.

9. The CFP of claim 8, wherein the carboxylic acid comprises propionic acid, 2-chloroacetic acid, phenylacetic acid, 4-chlorobenzoic acid, 2-fluorobenzoic acid, acrylic acid, butyric acid, 4-amino benzoic acid, acetyl salicylic acid, ethyl-l-hexanoic acid, formic acid anthralic acid, citric acid, maleic acid, malonic acid, 3-nitrobenzoic acid, oxalic acid, 4-pentenoic acid, tartaric acid, p-toluenesulfonic acid, phenyacetic acid, methanesulfonic acid, terephthalic acid, trimesic acid, and tannic acid.

10. The CFP of claim 1, wherein mixing of the first reactant and the second reactant comprises supplying the first reactant and the second reactant at a first molar equivalent ratio between the first reactant and the second reactant from about 0.25:1 to about 4:1.

11. The CFP of claim 10, wherein the first molar equivalent ratio or a second molar equivalent ratio is achieved by selecting a first flow rate of the first continuous flow of the first reactant and selecting a second flow rate of the second continuous flow of the second reactant based on the a first Molarity of the first reactant and a second Molarity of the second reactant.

12. The CFP of claim 1, wherein the reactor comprises a plug flow reactor (PFR), a perfectly mixed flow reactor, a continuously stirred tank reactor (CSTR), or a tube-in-tube reactor (TTR).

13. The CFP of claim 12, wherein the reactor comprises a PFR, and wherein the first continuous flow and the second continuous flow enter the PFR at a mixing location that is proximate a first end of the PFR; wherein the mixing location comprises a T-shaped junction or a Y-shaped junction connecting a path of the first continuous flow and a path of the second continuous flow to the PFR.

14. The CFP of claim 13, wherein the PFR further comprises a back-pressure valve located proximate a second end of the PFR, wherein the CFP further comprises maintaining a pre-selected pressure within the PFR via operation of the back-pressure valve.

15. The CFP of claim 12, wherein the reactor comprises a CSTR including (i) a first inlet operatively connected to the first continuous flow, (ii) a second inlet operatively connected to the second continuous flow, (iii) a product outlet, and (iv) an agitator.

16. The CFP of claim 15, wherein the CSTR further comprises a gas inlet operatively connected to a gas supply, wherein the gas inlet further comprises a pressure-control valve configured to maintain a pre-selected pressure within the CSTR.

17. A system for a continuous flow production of an acid chloride, comprising:
(i) a first reactant housed in a first vessel, the first reactant comprising a chlorine-donating compound;
(ii) a second reactant housed in a second vessel, the second reactant comprising a carboxylic acid;
(iii) a reactor, wherein the first vessel is operatively connected to the reactor via a first conduit and the second vessel is operatively connected to the reactor via a second conduit;
(iv) a product vessel operatively connected to an outlet of the reactor.

18. The system of claim 17, further comprising a pressure regulator configured to maintain a pre-selected pressure within the reactor.

19. The system of claim 18, further comprising one or more of:
a first heating device configured to supply heat to the first vessel, the second vessel, or both;
a second heating device configured to supply heat to the second vessel; and
a third heating device configured to supply heat to at least a portion of the reactor.

20. The system of claim 18, further comprising:
(v) a first pump configured to transfer the first reactant from the first vessel, through the first conduit, and into the reactor; and
(vi) a second pump configured to transfer the second reactant from the second vessel, through the second conduit, and into the reactor.
